(19) United States
(12) Patent Application Publication
(10) Pub. No.: US 2005/0085794 A1
(43) Pub. Date: Apr. 21, 2005

(54) Catheter connecting device

(76) Inventors: Patrick Denoth, Aarwangen (CH); Sandro Niederhaeuser, Bleienbach (CH); Astrid Valiquer, Villars-Ste-Croix (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 10/491,638

(22) PCT Filed: Sep. 23, 2002
(86) PCT No.: PCT/CH02/00529

(30) Foreign Application Priority Data
Oct. 5, 2001 (DE) 101 49 051.8

(51) Int. Cl. A61M 25/16

(52) U.S. Cl. 604/533

(57) ABSTRACT

The invention relates to a catheter connection comprising a support sleeve for fixing at least one catheter end and at least one attachment element to which the catheter end(s) can be attached, in addition to a connection structure, to which a clamping sleeve for fixing the catheter ends can be fastened. The invention also relates to a clamping sleeve, which can be pushed onto a catheter and has a tapered area, in addition to the individual elements of the catheter connection.

The present invention relates to a device for a catheter connection, in particular to an implantable connection for connecting two catheters or catheter ends for intravenous access, and to individual elements of the catheter connection.

A catheter connection for a subcutaneous port system is known from WO 99/53981, wherein one end of a catheter to be attached to a port body is slid onto a support of the port body and the slid-on catheter piece is pressed on using a sleeve and thus fastened to the port body. Fastening it directly to the port body, however, has the result that various hand operations are required on the port body itself, which is disruptive in particular when is just being implanted and is to be connected to the catheter, since these hand operations have to be performed in the immediate area of the open tissue.

It is an object of the present invention to provide a catheter connection which simplifies connecting a catheter to a port body.

This object is solved by a catheter connection and/or individual elements of the catheter connection as defined in the independent claims. Advantageous embodiments follow from the sub-claims.

In accordance with a first aspect of the invention, a support is provided for fastening at least one, preferably two or also three or more catheter ends or catheters, comprising at least one joining element to which the catheter element can be attached, for example slid onto. The joining element is preferably configured to be cylindrical and advantageously hollow on the inside, wherein the inner diameter of the joining element is advantageously about equally as large as the inner diameter of the catheter to be connected. Furthermore, a connecting structure is provided, on which a sleeve—which has preferably been slid onto the catheter before the catheter element is attached to or slid onto the catheter—can be fastened to the support. Once the catheter has been joined on, the sleeve slid onto the catheter is slid towards the catheter end to be connected, until the sleeve reaches the connecting structure of the support, such that the sleeve can be connected to the connecting structure and a fixed connection, preferably a non-positive-lock, positive-lock and/or frictional-lock connection, can thus be established, for example by pinching the catheter ends between the support and the sleeve.

The connecting structure to which the sleeve can be fastened is preferably a thread or threaded section, such that the sleeve can be screwed onto the support. Particularly advantageously, a structure for a latching or locking connection is provided in the area of the thread, for example adjacent to the thread, such as for example a groove encircling the support or a recess in sections, with which corresponding protrusions or latching hooks of the clamping sleeve can engage in order for example to secure the sleeve against being unintentionally released and in order to advantageously relieve the connection between the support and the clamping sleeve via the thread. The surface structure for the latching or locking connection can also be provided without a thread on the support.

A joining element of the support to which the catheter end is attached can for example comprise a circumferentially smooth, ribbed or structured surface, wherein a structured surface, for example one comprising a number of protrusions or small elevations, is advantageous in conjunction with a clamping sleeve for attaching or fastening a catheter end, for example by pinching. The joining element is preferably formed to be roughly tubular.

In accordance with another aspect of the present invention, a sleeve is provided which can be attached to a catheter end and preferably comprises a passage in order to be able to slide onto the catheter. The sleeve is preferably formed to be conical at least in sections, i.e. the outer diameter of the sleeve decreases from the side of the sleeve, which serves to connect to the support, towards the end of the sleeve, which points away from the support when connected. Such a conical configuration of a sleeve is advantageous when moving a catheter connection which is surrounded by tissue, since due to the conical configuration, surrounding tissue can be laterally displaced by the tapering end of the sleeve and guided around the connection.

Advantageously, the outer diameter of the sleeve at the end pointing away from the support decreases to a diameter which is roughly equal to the outer diameter of the cannula, such that the sleeve abuts the catheter on its outer area. However, the minimum outer diameter of the sleeve can also be formed such that the sleeve protrudes a certain distance of for example 0.1 to 2 mm from the upper side of the catheter, wherein the end of the sleeve is then advantageously configured such that no sharp edges protrude, which would act as cutting edges when the connection is shifted in tissue. Thus, the sleeve can for example be gently rounded at the end.

Particularly advantageously, an inner thread is provided on at least one end of the clamping sleeve and can be screwed onto the outer thread of a support. A latching or locking connection is preferably provided, for example in the form of a number of elastic latching hooks which can engage with a corresponding connecting element on the support, such as for example a circumferential groove, in order to be able to connect a clamping sleeve disposed on the support to the support as securely as possible.

In accordance with another aspect of the present invention, a catheter connection is disclosed, comprising a support as described above and at least one sleeve as described above, preferably at least two sleeves as described above, using which a connection can be established between two catheter ends.

The two ends of the support on which the clamping sleeves for fastening the catheter ends are disposed can be approximately identical or can be provided with different features, such as for example different threads or plugging and/or locking connections, in order to ensure that a particular clamping sleeve is unambiguously assigned to a particular end of the support.

Advantageously, the support and the at least one clamping sleeve are adjusted to each other such that a pressing or clamping connection can be established, for fastening a catheter end between the at least one clamping sleeve and the support, wherein said connection can reliably prevent the catheter from detaching from the support.

Individual components or all the components of the of the invention described above are preferably made of bio-compatible and/or dimensionally stable material, such as for example polyurethane, silicone, titanium or other suitable substances which are recognised as safe with respect to histocompatibility.

In accordance with another aspect, the present invention relates to a system comprising a catheter connection and a port body, to which a catheter piece is advantageously already fixedly attached before being inserted into tissue. Thus, once the port body has been inserted, it is only necessary to connect the catheter piece protruding from the port body to the catheter piece already inserted into the tissue using the catheter connection in accordance with the invention, wherein—providing the catheter ends to be connected are of a suitable length—the connection can be made out of the immediate vicinity of the open tissue. Since the sleeves are formed conically, the catheter connection can easily be shifted in the tissue.

The invention shall be described below on the basis of a preferred example embodiment. There is shown:

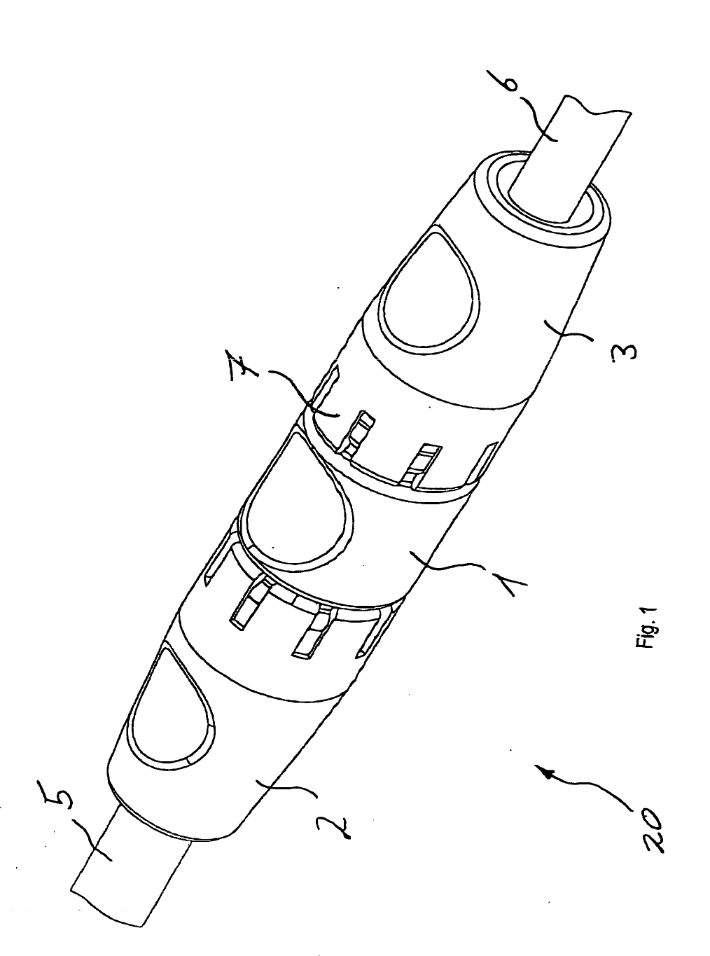

FIG. 1 a catheter connection in accordance with the invention, in a perspective view;

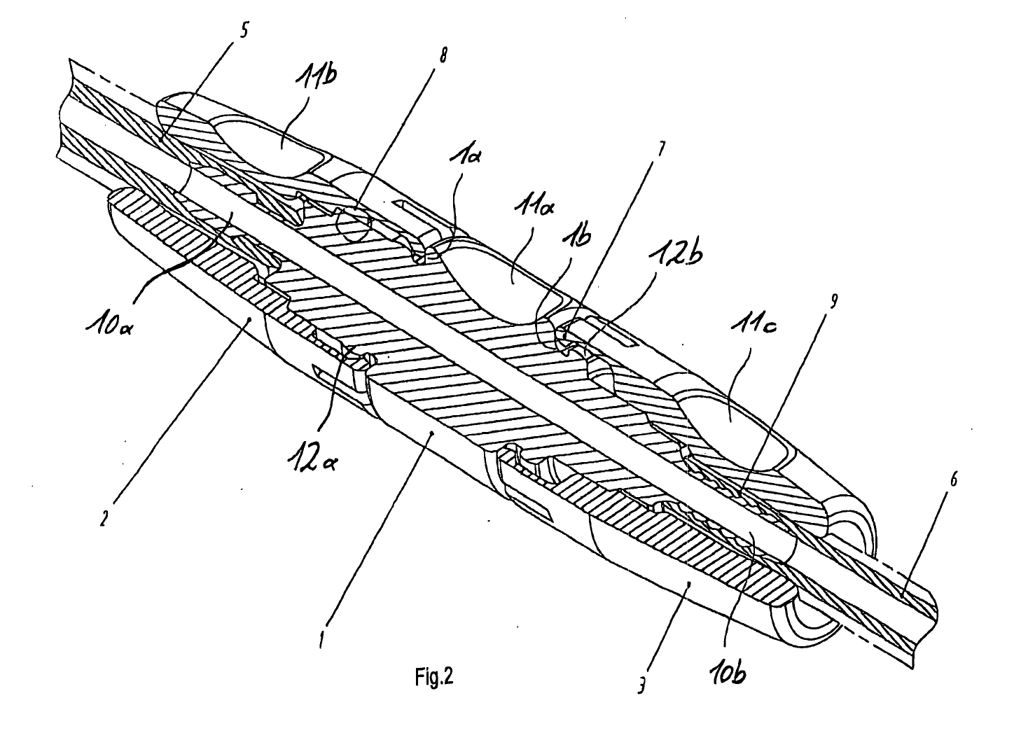

FIG. 2 a sectional view of the catheter connection shown in FIG. 1;

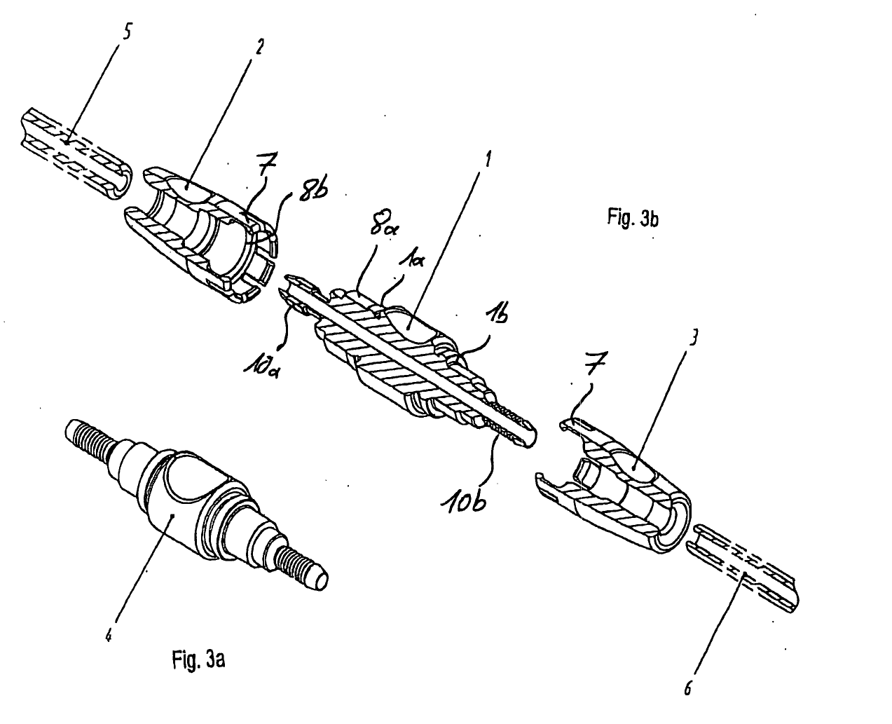

FIG. 3 a support in accordance with the invention; and

FIG. 3b an exploded view of a catheter connection in accordance with the invention, comprising another support and two clamping sleeves.

FIG. 1 shows a catheter connection in accordance with the invention, in a perspective representation, wherein a distal catheter end 5 is connected to a proximal catheter end 6 by the catheter connection 20.

FIG. 2 shows the catheter connection of FIG. 1 in a partial sectional view, wherein it can be seen that the distal catheter end 5 is placed onto the distal joining pipe 10a whose inner diameter is about equally as large as the inner diameter of the catheter 5. The distal joining pipe 10a of the support 1 is formed so as to conically taper towards the distal end of the support 1, in order to simplify joining on the distal catheter 5. The distal joining pipe 10a gently expands towards the proximal end and has a slightly larger outer diameter than at the proximal end, in order to enable the distal catheter 5 to be firmly clamped by the expanded area of the distal joining pipe 10a.

Before the distal catheter 5 is joined onto the support 1, the distal clamping sleeve 2 is slid onto the catheter 5. If the catheter 5 has been slid onto the joining pipe 10a, then the clamping sleeve 2 can be shifted along the catheter 5 towards the support 1. In the area encircling the support 1, indicated by 8, an inner thread 8b of the clamping sleeve 2—shown in FIG. 3b—engages with an outer thread 8a of the support 1, whereby the clamping sleeve 2 can be screwed onto the support 1′ until the latching hooks 7 provided on the end of the clamping sleeve 2 facing the support 1 can engage with a holding groove 1a of the support 1, in order to establish a locking or elastic block of the clamping sleeve 2 on the support 1, which can relieve the threaded connection 8 and prevent the clamping sleeve 2 from being unintentionally detached from the support 1, for example by inadvertently twisting open the threaded lock 8. The locking connection is realised by a number of, e.g. eight, latching hooks 7 provided around the circumference of the clamping sleeve 2, which are formed on elongated protruding sections of the clamping sleeve 2, such that the latching hooks 7 can spring in the radial direction of the clamping sleeve 2, so as to travel over the annular protrusion protruding in front of the holding groove 1a and then to engage with the holding groove 1a, when the clamping sleeve 2 is twisted onto the support sleeve 1.

A proximal joining pipe 10b is provided at the proximal end of the support 1, wherein the proximal catheter end 6 can be slid onto said proximal joining pipe 10b. The upper side of the proximal joining pipe 10b comprises a ribbed structure formed on the joining pipe 10b by a number of circumferential expansions in a row, so as to simplify joining and clamping the proximal catheter end 6 on the proximal joining pipe 10b. The proximal clamping sleeve 3, just like the distal clamping sleeve 2 described above, is firstly slid onto the proximal catheter 6. Then the catheter 6 is placed onto the joining pipe 10b and together with the clamping sleeve 3 slid over it clamped between the joining pipe 10b and the clamping sleeve 3 by screwing on and locking the clamping sleeve 3 on the holding groove 1b, by means of the latching hooks 7.

As may be seen from FIG. 3a, the support sleeve 1 shown in FIG. 3b can also—in an alternative embodiment—comprise other configurations of the joining pipes 10a and 10b, which in the example shown both exhibit a grooved surface structure. Generally speaking, any combination of identical or different configurations of the distal and proximal joining pipes 10a and 10b can be used, which for example can exhibit a smooth, ribbed or structure surface.

To simplify handling, holding areas 11a, 11 and 11c are formed on the support sleeve 1 and the clamping sleeves 2 and 3, which simplify holding and rotating the support sleeve 1 and the clamping sleeves 2 and 3.

To disconnect the catheters 5 and 6, the procedure described above is performed in reverse order, i.e. firstly, the locking or latching connection of the latching hooks 7 in the corresponding holding groove 1a or 1b is released by exerting a pressure on the elastic elements 7 in the area facing the main body of the respective clamping sleeve 2 or 3, i.e. in the area of the side of the latching hook 7 facing the clamping sleeve 2 or 3, which raises the front end of the respective latching hook 7 outwards out of the respective holding groove 1a or 1b by a leverage force transferred via the elevation 12a or 12b acting as a support point for the lever, so as to release the latching or locking connection. The clamping sleeve 2, 3 can then be unscrewed from the support 1 and slid back along the catheter 5, 6, in order to release the clamping connection of the corresponding catheter 5, 6 on the joining pipe 10a, 10b. The catheter 5, 6 can then be removed from the joining pipe 10a, 10b and the clamping sleeve 2, 3 from the catheter 5, 6. The catheter connection formed by the catheter connection in accordance with the invention can thus be easily and reversibly connected and released.

What is not shown in the figures is a percutaneous port system which is preferably situated in the vicinity of the catheter connection 20 in accordance with the invention, for example offset approx. 5 cm from it. The catheter connection 20 can then be provided in the same subcutaneous skin pocket as the port body, wherein the port body can for example already be fixedly connected to the proximal catheter 6 before insertion and no longer has to be connected to the proximal catheter 6 after insertion.

Various infusion solutions, such as for example a nutritional solution, various medicinal solutions, cytostatics or various rinsing solutions, can be introduced into the desired area of the body via the proximal catheter 6, through the catheter connection 20 in accordance with the invention, to the distal catheter 5. If the inner diameter of the support is formed to be about as large as the inner diameter of the catheter to be connected, then a laminar flow of a medium can be ensured due to the substantially constant cross-section.

Consequently, the catheter connection 20 in accordance with the invention can be simply attached without any appreciable tensile stresses on the distal or proximal catheters 5, 6 and, due to the conical shaping in the area of the clamping sleeves 2 and 3, can be relatively easily moved through surrounding tissue with little danger of injury to the tissue, if the catheter connection 20 is to be removed from the tissue or slid to a particular area of the tissue.

1-14. (canceled)

15. A support for fastening at least one catheter end comprising at least one joining element onto which said at least one catheter end can be placed, and at least one connecting structure to which a sleeve for fastening the at least one catheter end can be fastened.

16. The support as set forth in claim 15, wherein from two to ten joining elements are provided on said support.

17. The support as set forth in claim 15, wherein said connecting structure is an outer thread or threaded section of the support sleeve.

18. The support as set forth in claim 15, wherein the connecting structure is a surface structure for a latching or locking connection, in particular a holding groove.

19. The support as set forth in claim 15, wherein the at least one joining element is formed to be roughly tubular and/or comprises a smooth, ribbed or structured surface on its outer circumference.

20. A sleeve which can be slid onto a catheter and comprises a conically tapering area.

21. The sleeve as set forth in claim 20, wherein said conically tapering area tapers to approximately the outer circumference of a catheter.

22. The sleeve as set forth in claim 20, wherein the sleeve further comprises an inner thread.

23. The sleeve as set forth in claim 20, further comprising at least one connecting element carried on the sleeve.

24. The sleeve as set forth in claim 23, wherein said at least connecting element is carried elastically on the sleeve.

25. A catheter connection comprising:
at least one support for fastening at least one catheter end, said at least one support comprising at least one joining element onto which said at least one catheter end can be placed, and at least one connecting structure to which a sleeve for fastening the at least one catheter end can be fastened; and
at least one sleeve which can be slid onto a catheter and comprises a conically tapering area.

26. The catheter connection as set forth in claim 25, comprising two joining elements and two corresponding sleeves.

27. The catheter connection according to claim 20, wherein at least one of the at least one support and the at least one sleeve are formed at least partially from a bio-compatible material.

28. The catheter connection according to claim 27, wherein the bio-compatible material is selected from the group comprising polyurethane, silicone and titanium.

29. A catheter system comprising a catheter and a port body, said catheter system comprising:
at least one support for fastening at least one catheter end, said at least one support comprising at least one joining element onto which said at least one catheter end can be placed, and at least one connecting structure to which a sleeve for fastening the at least one catheter end can be fastened;
at least one sleeve which can be slid onto the catheter and comprises a conically tapering area.

30. A connecting device for use with a catheter, said device comprising:
a connecting structure; and
a sleeve received on the catheter, wherein the sleeve is moved along the catheter toward the connecting structure until the sleeve is connected to the connecting structure thereby capturing the catheter between the connecting structure and the sleeve.

31. The connecting device according to claim 30, wherein the sleeve is conical.

32. The connecting device according to claim 31, wherein the sleeve is conical, one end of the sleeve having a smaller outer diameter than the other end, the end with the smaller outer diameter being rounded.
